(19) United States
(12) Patent Application Publication
(10) Pub. No.: US 2015/0337308 A1
(43) Pub. Date: Nov. 26, 2015

(54) MATRIX METALLOPROTEINASE 9 (MMP-9) APTAMER AND USES THEREOF

(71) Applicants: INSERM (INSTITUT NATIONAL DE LA SANTE ET DE LA RECHERCHE MEDICALE), Paris (FR); UNIVERSITE DE BORDEAUX, Bordeaux (FR); CHU DE BORDEAUX, Talence Cedex (FR)

(72) Inventors: Jean-Jacques Toulme, Cedex Bordeaux (FR); Sonia Da Rocha, Cedex Pessac (FR); Eric Dausse, Cedex Bordeaux (FR); Michele Allard, Cedex Bordeaux (FR); Laurent Azema, Cedex Bordeaux (FR)

(73) Assignee: CHU DE BORDEAUX, Talence Cedex (FR)

(21) Appl. No.: 14/391,408

(22) PCT Filed: Apr. 11, 2013
(86) PCT No.: PCT/EP2013/057529

§ 371 Date: Oct. 9, 2014

(30) Foreign Application Priority Data
Apr. 11, 2012 (EP) 12305425.6

(51) Int. Cl. C12N 15/115; A61K 49/00; A61K 51/04

(52) U.S. Cl. CPC C12N 15/115; A61K 51/0491; A61K 49/0002

(57) ABSTRACT

The present invention relates to a nucleic acid aptamer that binds specifically to human matrix metalloproteinase 9 (h MMP-9) and its use for imaging h MMP-9 in a subject in need thereof.

### FIELD OF THE INVENTION

The present invention relates to a nucleic acid aptamer that binds specifically to the matrix metalloproteinase 9 (MMP-9) and uses thereof for imaging hMMP-9 in a subject.

### BACKGROUND OF THE INVENTION

Uncontrolled cellular proliferation is a cardinal feature of neoplasia. The ability to measure the proliferation rate in tumors in patients in vivo will help with tumor grading and staging, and assessing the effect of therapy. Today, modern imaging techniques enable the investigation of pathological changes within tumors themselves in vivo, exploiting the increased cellular metabolism in these cells using Positon Emission Tomography (PET) imaging of 3′-déoxy-3′-[¹⁸F]fluorothymidine (FLT), [¹¹C]-methionine or O-2-[¹⁸F]-fluoroethyl-tyrosine (FET). New imaging modalities are required for a better monitoring of tumor malignity associated with extra cellular damage in surrounding tissue, especially functional imaging reflecting intimate biological mechanisms of the tumor cells proliferation.

One of the possible mechanisms involved in surrounding tissue invasion is the overexpression of matrix metalloproteinases (MMPs) capable of degrading extra cellular matrix components, permitting cell migration (5-6). An alternative approach for imaging tumors would be to follow MMPs expression as a surrogate marker of malignant tumor cell invasion (7). For example, tumor cells form mass lesions in the central nervous system and enzymatic degradation of extra cellular matrix by MMPs are necessary for the malignant tumor cells to migrate into normal brain tissue, and MMP inhibitors are attractive potential anti-cancer agents (8-9).

hMMP-2 and hMMP-9 constitute a subgroup of MMPs called gelatinases that degrade the basal lamina around capillaries, and enable angiogenesis and neurogenesis, participating in extra cellular matrix degradation and facilitating tumor cells migration. Indeed, abnormal expression of MMP-9 has been associated with tumor progression. Notably, over-expression of MMP-9 has been shown to be linked to progression of meningiomas (See notably Paek et al., 2006, Oncol Rep, Vol. 16(1): 49-56; Okuducu et al., 2006, Cancer, Vol. 107(6): 1365-1372; Okuducu et al., 2006, Vol. 48(7): 836-845). Over-expression of MMP-9 has also been measured in patients affected with breast cancer, with bladder tumors and with colorectal cancer (See Somiari et al., 2006, Int J Cancer, Vol. 119(6): 1403-1411; Di Carlo et al., 2006, Oncol Rep, Vol. 15(5): 1321-1326; Ogata et al., 2006, Cancer Chemother Pharmacol, Vol. 57(5): 577-583). Also, invasive macroprolactinomas were found significantly more likely to express MMP-9 than non-invasive macroprolactinomas. Accordingly, MMP-9 is a relevant marker of malignant tumors.

Aptamers have been developed in the early 90's (11-13). These structured DNA, RNA or modified oligonucleotides are identified after iterative cycles of selection/amplification through a process named SELEX (Systematic Evolution of Ligands by Exponential enrichment) from a random oligonucleotide library. Aptamers have been successfully selected for a wide range of targets (proteins, nucleic acids, peptides, small molecules, cells . . . ) and were shown to display both high affinity and specificity (14-15). Aptamer-based tools were designed for diagnostic or therapeutic applications over the last decade and are a promising alternative to monoclonal antibodies in many applications (16-17) including molecular imaging (18). Aptamers can be modified for making them resistant to nucleases and conjugated to fluorescent tags or radioelements. The first aptamer for in vivo imaging was developed in 1997 for the detection of human neutrophil elastase in a rat model of inflammation (19). Since these encouraging results, aptamers have been successfully applied to target tumor cells for detection or real-time imaging (20-27). Most of aptamer imaging probes have been selected against cells for cancer detection more particularly with aptamer-conjugated nanoparticles (23,28-30). Recently, an antibody-like nanostructure composed of two aptamers and a dendrimer was developed with temperature-dependent binding to cancer cells (31). Histological analyses have been carried out with fluorescent or biotinylated aptamers (32-36). A new strategy based on activable aptamer showed less fluorescence background with specific tumor retention (37).

### SUMMARY OF THE INVENTION

The present invention relates to a nucleic acid aptamer that binds specifically to human matrix metalloproteinase 9 (hMMP-9) characterized in that said nucleic acid comprises the following nucleotide sequence:

5′-NS1-CCU-NS3-N1-NS5-CCCGUUAGCC-NS6-N2-NS4-
CCC-NS2-3′

wherein
NS1 and NS2 consist of polynucleotides having 1, 2 or 3 nucleotides in length, and NS1 and NS2 have complementary sequences;
NS3 and NS4 consist of polynucleotides having 2 nucleotides in length, and NS1 and NS2 have complementary sequences
N1 and N2 consist of a nucleotide, and N1 is or is not complementary to N2
NS5 and NS6 consist of polynucleotides having 4 nucleotides in length, and NS5 and NS6 have complementary sequences.

### DETAILED DESCRIPTION OF THE INVENTION

For the present invention, the inventors selected an RNA aptamer containing 2′fluoro, pyrimidine ribonucleosides, that exhibits a strong affinity for hMMP-9 (Kd=20 nM) and that discriminates other human MMPs: no binding was detected to either hMMP-2 or -7. Investigating the binding properties of different MMP-9 nucleic acid aptamer variants by surface plasmon resonance allowed the determination of recognition elements. As a result, a truncated aptamer, 36 nucleotide long was made fully resistant to nuclease following the substitution of every purine ribonucleoside residue by 2′-O-methyl analogues and was conjugated to S-acetylmercaptoacetyltriglycine for imaging purposes. The resulting modified aptamer retained the binding properties of the originally selected sequence. Following ^{99m}Tc labelling this aptamer was used for ex vivo imaging slices of human brain tumors. The inventors were able to specifically detect the presence of hMMP-9 in such tissues.

Accordingly a first object of the present invention relates to a nucleic acid aptamer that binds specifically to human matrix metalloproteinase 9 (hMMP-9) characterized in that said nucleic acid comprises the following nucleotide sequence:

5′-NS1-CCU-NS3-N1-NS5-CCCGUUAGCC-NS6-N2-NS4-
CCC-NS2-3′

wherein
NS1 and NS2 consist of polynucleotides having 1, 2 or 3 nucleotides in length, and NS1 and NS2 have complementary sequences;
NS3 and NS4 consist of polynucleotides having 2 nucleotides in length, and NS1 and NS2 have complementary sequences
N1 and N2 consist of a nucleotide, and N1 is or is not complementary to N2
NS5 and NS6 consist of polynucleotides having 4 nucleotides in length, and NS5 and NS6 have complementary sequences

As used herein, a “nucleotide” is selected from the group consisting of A, T, U, G or C, and any chemically modified form thereof.

In every hMMP-9 nucleic acid aptamer according to the invention, the various “NS” sequences are included in a stem secondary structure, with a given first NS sequence being complementary to a given second NS sequence. Thus, when present in the nucleic acid sequence of a hMMP-9 nucleic acid aptamer according to the invention, (i) NS1 and NS2 are complementary and form together a double-stranded stem secondary structure, as it is the case also for (ii) NS3 and NS4, and (iii) NS5 and NS6. The specific nucleic acid sequence of a given NSx sequence is not essential, provided that the base pair complementarity between two given NSx sequences is ensured for forming the corresponding stem region of the hMMP-9 nucleic acid aptamer under consideration.

In one embodiment, NS1 represents C, GC (SEQ ID NO: 13), UGC (SEQ ID NO:14) or ACG (SEQ ID NO: 15) and accordingly NS2 represents G, GC (SEQ ID NO:13), GCA (SEQ ID NO:16) or CGU (SEQ ID NO:17) respectively.

In one embodiment, NS3 and NS4 represent GC (SEQ ID NO: 13) or CG (SEQ ID NO:18).

In one embodiment N1 and N2 represent C or A. In another embodiment N1 represents C and N2 represents G.

In one embodiment, NS5 represents CUCA (SEQ ID NO:19) or GAGU (SEQ ID NO:20) and accordingly NS6 represents UGAG (SEQ ID NO:21) or ACUC (SEQ ID NO:22) respectively.

In another particular embodiment, the nucleic acid aptamer of the invention comprises or consists of a nucleic acid sequence selected from the group consisting of:

(SEQ ID NO: 1)
F3C1: CCCUGCCCUCACCCGUUAGCCUGAGCGCCCCG
(SEQ ID NO: 2)
F3B′: GCCCUGCCCUCACCCGUUAGCCUGAGCGCCCCGC
(SEQ ID NO: 3)
F3B: UGCCCUGCCCUCACCCGUUAGCCUGAGCGCCCCGCA
(SEQ ID NO: 4)
F3BAA: UGCCCUGCACUCACCCGUUAGCCUGAGAGCCCCGCA
(SEQ ID NO: 5)
F3BCG: UGCCCUGCCCUCACCCGUUAGCCUGAGGGCCCCGCA
(SEQ ID NO: 6)
F3Binv1: UGCCCUGCCGAGUCCCGUUAGCCACUCCGCCCCGCA
(SEQ ID NO: 7)
F3Binv2: ACGCCUCGCCUCACCCGUUAGCCUGAGCCGCCCCGU.
In one embodiment, the nucleic acid aptamer of
the invention consists of F3B:
(SEQ ID NO: 3)
UGCCCUGCCCUCACCCGUUAGCCUGAGCGCCCCGCA

In certain other embodiments, the nucleic acid sequence of such a hMMP-9 nucleic acid aptamer comprises a nucleic acid sequence as above described, and thus also comprises either (i) one additional nucleic acid sequence located at the 5′-end or at the 3′-end of the said aptamer or (ii) one additional nucleic acid sequence located at each of both the 5′-end and the 3′-end of the said aptamer. These additional nucleic acid sequences may have from 1 to 32 nucleotides in length, irrespective of the identity of the added sequence(s), without significantly altering the binding properties of the resulting aptamer to hMMP-9. Thus, these additional nucleic acid sequences may have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or 32 nucleotides in length, while maintaining binding properties similar to the binding properties of the corresponding hMMP-9 nucleic acid aptamer without the additional sequence(s), i.e. a (KD) dissociation constant which is at most distinct of one order of magnitude, as compared with the corresponding hMMP-9 nucleic acid aptamer without the additional sequence(s).

As shown in the examples herein, an illustrative hMMP-9 nucleic acid aptamers as set forth in SEQ ID NO:8 (F3) comprise 16-mer additional sequences located at the 5′-end and 16-mer additional sequences located at the 3′-end of the nucleic acid sequences as set forth in SEQ ID NO:2, while having binding properties which are similar with, if not identical to, the binding properties of the corresponding hMMP-9 nucleic acid aptamers wherein these additional sequences are absent. The additional sequences may form secondary structure(s) of internal loop(s), stem(s), or both.

According to another particular embodiment, the nucleic acid aptamer of the invention thus comprises or consists of a nucleic acid sequence as set forth in SEQ ID NO:8 (F3: GGUUACCAGCCUUCACUGCCCUGCCCUCACCCGUUAGCCUGAGCGCCCCGCACC ACGGUCGGUCACAC).

Nucleic acids of the invention may be produced by any technique known in the art, such as, without limitation, any chemical, biological, genetic or enzymatic technique, either alone or in combination. Knowing the nucleic acid sequence of the desired sequence, one skilled in the art can readily produce said aptamers, by standard techniques for production of polynucleotides. For instance, they can be synthesized using well-known solid phase method, preferably using a commercially available polynucleotide synthesis apparatus.

In preferred embodiments, any one of the hMMP-9 nucleic acid aptamers according to the present invention may be chemically modified, so as to increase its chemical stability both in vitro and in vivo, and notably so as to decrease its degradation by cellular enzymes, typically its degradation by exonucleases and endonucleases. Chemically modified hMMP-9 nucleic acid aptamers are particularly suitable for their use in vivo, either as such or combined with active compounds like protease inhibitors for medical purposes.

One potential problem encountered in the use of nucleic acids is that oligonucleotides in their phosphodiester form may be quickly degraded in body fluids by intracellular and extracellular enzymes such as endonucleases and exonucleases before the desired effect is manifest. The SELEX™ method (i.e. U.S. Pat. No. 5,270,163) thus encompasses the identification of high-affinity nucleic acid ligands containing modified nucleotides conferring improved characteristics on the ligand, such as improved in vivo stability or improved delivery characteristics. Examples of such modifications include chemical substitutions at the sugar and/or phosphate and/or base positions. SELEX™ identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH.sub.2), 2′-fluoro (2′-F), and/or 2′-OMe substituents. Techniques 2′-chemical modification of nucleic acids are also described in the US patent applications No US 2005/0037394 and No US 2006/0264369.

Modifications of the nucleic acid ligands contemplated in this invention include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Modifications to generate oligonucleotide populations which are resistant to nucleases can also include one or more substituted internucleotide linkages, altered sugars, altered bases, or combinations thereof. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil, backbone modifications, phosphorothioate or alkyl phosphate modifications, methylations, and unusual base-pairing combinations such as the isobases isocytidine and isoguanidine. Modifications can also include 3′ and 5′ modifications such as capping.

In one embodiment, oligonucleotides are provided in which the P(O)O group is replaced by P(O)S (“thioate”), P(S)S (“dithioate”), P(O)NR.sub.2 (“amidate”), P(O)R, P(O)OR′, CO or CH.sub.2 (“formacetal”) or 3′-amine (—NH—CH.sub.2--CH.sub.2--), wherein each R or R′ is independently H or substituted or unsubstituted alkyl. Linkage groups can be attached to adjacent nucleotides through an —O—, —N—, or —S— linkage. Not all linkages in the oligonucleotide are required to be identical. As used herein, the term phosphorothioate encompasses one or more non-bridging oxygen atoms in a phosphodiester bond replaced by one or more sulfur atoms

In further embodiments, the oligonucleotides comprise modified sugar groups, for example, one or more of the hydroxyl groups is replaced with halogen, aliphatic groups, or functionalized as ethers or amines. Alternatively the oligonucleotides comprise LNA (Locked Nucleic Acid), FANA (Fluoro Arabino Nucleic Acid), and derivatives of locked or acyclic sugars. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al., Nucl. Acid Res. 19:2629-2635 (1991); and Hobbs, et al, Biochemistry 12:5138-5145 (1973). Other modifications are known to one of ordinary skill in the art. Such modifications may be pre-SELEX™ process modifications or post-SELEX™ process modifications (modification of previously identified unmodified ligands) or may be made by incorporation into the SELEX™ process.

Pre-SELEX™ process modifications or those made by incorporation into the SELEX™ process yield nucleic acid ligands with both specificity for their SELEX™ target and improved stability, e.g., in vivo stability. SELEX™ process modifications made to nucleic acid ligands may result in improved stability, e.g., in vivo stability without adversely affecting the binding capacity of the nucleic acid ligand.

The SELEX™ method encompasses combining selected oligonucleotides with other selected oligonucleotides and non-oligonucleotide functional units as described in U.S. Pat. No. 5,637,459 and U.S. Pat. No. 5,683,867. SELEX™ method further encompasses combining selected nucleic acid ligands with lipophilic or non-immunogenic high molecular weight compounds in a diagnostic or therapeutic complex, as described, e.g., in U.S. Pat. No. 6,011,020, U.S. Pat. No. 6,051,698, and PCT Publication No. WO 98/18480. These patents and applications teach the combination of a broad array of shapes and other properties, with the efficient amplification and replication properties of oligonucleotides, and with the desirable properties of other molecules.

Thus, in certain embodiments of the hMMP-9 nucleic acid aptamers according to the invention, the said hMMP-9 nucleic acid aptamers are protected against hydrolysis by nucleases by chemical modification.

In one embodiment, all pyrimidines are 2′-fluoropyrimidine. Accordingly, the present invention relates to a nucleic acid aptamer that binds specifically to human matrix metalloproteinase 9 (hMMP-9) comprising or consisting of nucleic acid sequence selected from the group consisting of SEQ ID NO:1-8 wherein the pyrimidines are replaced by 2′-fluoropyrimidines.

In one embodiment, the nucleic acid aptamer of the invention consists of F3B sequence (UGCCCUGCCCUCACCCGUUAGCCUGAGCGCCCCGCA (SEQ ID NO: 3) wherein the pyrimidines are 2′-fluoropyrimidine and the purine ribonucleosides are substituted by 2′O-methyl residues (F3Bomf).

The present invention is also directed to the use of the hMMP-9 nucleic acid aptamers of the present invention for the molecular imaging of hMMP-9 and the diagnosis of pathophysiological conditions associated with hMMP-9. In particular, the invention encompasses imaging agents, kits and strategies for specifically detecting the presence of hMMP-9 in vitro, ex vivo as well as in vivo using imaging techniques.

In one aspect, the invention relates to a new class of imaging agents that have high affinity and specificity for hMMP-9. More specifically, hMMP-9-targeted imaging agents are provided that comprise at least one hMMP-9 nucleic acid aptamer moiety of the invention associated with at least one detectable moiety.

The term “detectable moiety”, as used herein refers to any entity which, when part of a molecule, allows visualization of the molecule, for example using imaging techniques.

In the context of the present invention, detectable moieties are entities that are detectable by imaging techniques such as ultrasonography, Magnetic Resonance Imaging (MRI), Positron Emission Tomography (PET), Single Photon Emission Computed Tomography (SPECT), fluorescence spectroscopy, Computed Tomography, X-ray radiography, or any combination of these techniques. Preferably, detectable moieties are stable, non-toxic entities which, when part of a hMMP-9-targeted imaging agent, retain their properties under in vitro and in vivo conditions.

In certain embodiments, the hMMP-9-targeted imaging agent is designed to be detectable by a nuclear medicine imaging techniques such as planar scintigraphy (PS), Positron Emission Tomography (PET) and Single Photon Emission Computed Tomography (SPECT). In such embodiments, the imaging agent of the invention comprises at least one hMMP-9 nucleic acid aptamer moiety associated with at least one radionuclide (i.e., a radioactive isotope). SPECT and PET techniques acquire information on the concentration of radionuclides introduced into a biological sample or a patient's body. PET generates images by detecting pairs of gamma rays emitted indirectly by a positron-emitting radionuclide. A PET analysis results in a series of thin slice images of the body over the region of interest (e.g., brain, breast, liver). These thin slice images can be assembled into a three dimensional representation of the examined area. However, there are only few PET centers because they must be located near a particle accelerator device that is required to produce the short-lived radioisotopes used in the technique. SPECT is similar to PET, but the radioactive substances used in SPECT have longer decay times than those used in PET and emit single instead of double gamma rays. Although SPECT images exhibit less sensitivity and are less detailed than PET images, the SPECT technique is much less expensive than PET and offers the advantage of not requiring the proximity of a particle accelerator. Planar scintigraphy (PS) is similar to SPECT in that it uses the same radionuclides. However, PS only generates 2D-information.

Thus, in certain embodiments, the at least one detectable moiety in an imaging agent of the invention is a radionuclide detectable by PET. Examples of such radionuclides include carbon-11 (¹¹C), nitrogen-13 (¹³N), oxygen-15 (¹⁵O) and fluorine-18 (¹⁸F).

In other embodiments, the detectable moiety is a radionuclide detectable by planar scintigraphy or SPECT. Examples of such radionuclides include technetium-99m (^{99m}Tc), gallium-67 (⁶⁷Ga), yttrium-91 (⁹¹Y), indium-111 (¹¹¹In)rhenium-186 (¹⁸⁶Re), and thallium-201 (²⁰¹Tl). Over 85% of the routine nuclear medicine procedures that are currently performed use radiopharmaceutical methodologies based on ^{99m}Tc. Therefore, in certain preferred embodiments, the at least one detectable moiety of an imaging agent is ^{99m}Tc.

In certain embodiments, the hMMP-9-targeted imaging agent is designed to be detectable by Magnetic Resonance Imaging (MRI). MRI, which is an application of Nuclear Magnetic Resonance (NMR), has evolved into one of the most powerful non-invasive techniques in diagnostic clinical medicine and biomedical research. It is widely used as a non-invasive diagnostic tool to identify potentially maleficent physiological anomalies, to observe blood flow or to determine the general status of the cardiovascular system. MRI has the advantage (over other high-quality imaging methods) of not relying on potentially harmful ionizing radiation.

Thus, in certain embodiments, an imaging agent of the invention comprises at least one hMMP-9 nucleic acid aptamer moiety associated with at least one paramagnetic metal ion. Examples of paramagnetic metal ions detectable by MRI are gadolinium III (Gd³⁺), chromium III (Cr³⁺), dysprosium III (Dy³⁺), iron III (Fe³⁺), manganese II (Mn²⁺), and ytterbium III (Yb³⁺). In certain preferred embodiments, the paramagnetic metal ion is gadolinium III (Gd³⁺). Gadolinium is an FDA-approved contrast agent for MRI.

In other embodiments, the imaging agent of the invention comprises at least one hMMP-9 nucleic acid aptamer moiety associated with at least one ultrasmall superparamagnetic iron oxide (USPIO) particle. USPIO particles are currently under investigation as contrast agents for imaging human pathologies (C. Corot et al., Adv. Drug Deliv. Rev., 2006, 56: 1472-1504). They are composed of a crystalline iron oxide core containing thousands of iron atoms which provide a large disturbance of the Magnetic Resonance signal of surrounding water. In contrast to other types of nanoparticles such as quantum dots (currently under investigation as extremely sensitive fluorescent probes), USPIO particles exhibit a very good biocompatibility. Chemical coating of USPIO particles is required to ensure their dispersion in biological media. The presence of an appropriate coating may also result in a decrease in the clearance of the particles (“stealth” effect) and may provide a means to bind these particles to molecules that are able to target a specific tissue (R. Weissleder et al., Magn. Reson. Q, 1992, 8: 55-63). Polysaccharides, such as dextran and its carboxymethylated derivatives, are currently used as coatings. USPIO particles are known in the art and have been described (see, for example, J. Petersein et al., Magn. Reson. Imaging Clin. Am., 1996, 4: 53-60; B. Bonnemain, J. Drug Target, 1998, 6: 167-174; E. X. Wu et al., NMR Biomed., 2004, 17: 478-483; C. Corot et al., Adv. Drug Deliv. Rev., 2006, 58: 1471-1504; M. Di Marco et al., Int. J. Nanomedicine, 2007, 2: 609-622). USPIO particles are commercially available, for example, from AMAG Pharmaceuticals, Inc. under the tradenames Sinerem® and Combidex®. The present invention proposes to coat USPIO particles with hMMP-9 nucleic acid aptamer moieties and use the resulting imaging agents to detect hMMP-9 by MRI.

In certain embodiments, the hMMP-9-targeted imaging agent is designed to be detectable by fluorescence spectroscopy. In such embodiments, the imaging agents of the invention comprise at least one hMMP-9 nucleic acid aptamer moiety associated with at least one fluorescent moiety.

Favorable optical properties of fluorescent moieties to be used in the practice of the present invention include high molecular absorption coefficient, high fluorescence quantum yield, and photostability. Preferred fluorescent moieties exhibit absorption and emission wavelengths in the visible (i.e., between 400 and 700 nm) or the near infra-red (i.e., between 700 and 950 nm). Selection of a particular fluorescent moiety will be governed by the nature and characteristics of the illumination and detection systems used in the diagnostic method. In vivo fluorescence imaging uses a sensitive camera to detect fluorescence emission from fluorophores in whole-body living mammals. To overcome the photon attenuation in living tissue, fluorophores with emission in the near-infrared (NIR) region are generally preferred (J. Rao et al., Curr. Opin. Biotechnol., 2007, 18: 17-25). The list of NIR probes continues to grow with the recent addition of fluorescent organic, inorganic and biological nanoparticles. Recent advances in imaging strategies and reporter techniques for in vivo fluorescence imaging include novel approaches to improve the specificity and affinity of the probes, and to modulate and amplify the signal at target sites for enhanced sensitivity. Further emerging developments are aiming to achieve high-resolution, multimodality and lifetime-based in vivo fluorescence imaging.

Numerous fluorescent moieties with a wide variety of structures and characteristics are suitable for use in the practice of the present invention. Suitable fluorescent labels include, but are not limited to, quantum dots (i.e., fluorescent inorganic semiconductor nanocrystals) and fluorescent dyes such as Texas red, fluorescein isothiocyanate (FITC), phycoerythrin (PE), rhodamine, fluorescein, carbocyanine, Cy-3™ and Cy-5™ (i.e., 3- and 5-N,N′-diethyltetra-methylindodicarbocyanine, respectively), Cy5.5, Cy7, DY-630, DY-635, DY-680, and Atto 565 dyes, merocyanine, styryl dye, oxonol dye, BODIPY dye (i.e., boron dipyrromethene difluoride fluorophore), Alexa Fluor dyes (e.g. Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750) and any fluorogenic related label, analogues, derivatives or combinations of these molecules.

In certain embodiments, the detectable moiety is detectable by time-resolved fluorometry. For example, the detectable moiety is europium (Eu³⁺).

As will be understood by one skilled in the art, the selection of a particular type of detectable moiety in the design of a hMMP-9-targeted imaging agent will be dictated by the intended purpose of the imaging agent as well as by the imaging technique to be used in the detection.

In certain embodiments, an imaging agent of the present invention may be designed to be detectable by more than one imaging technique, for example by a combination of MRI-PET, MRI-SPECT, fluorescence-MRI, X-ray radiography-scintigraphy, and the like. Multimodal imaging provides different types of information about biological tissues, such as both structural and functional properties. Thus, for example, an imaging agent according to the present invention may comprise at least one hMMP-9 nucleic acid aptamer moiety associated with at least one detectable moiety that is detectable by more than one imaging technique. Examples of such detectable moieties include, but are not limited to, europium, which is fluorescent and detectable by MRI; and luminescent hybrid nanoparticles with a paramagnetic Gd₂O₃ core that are developed as contrast agents for both in vivo fluorescence and MRI (J. L; Bridot et al., J. Am. Chem. Soc., 2007, 129: 5076-5084) Alternatively, an imaging agent may comprise at least one hMMP-9 nucleic acid aptamer moiety associated with a first detectable moiety and a second detectable moiety, wherein the first detectable moiety is detectable by a first imaging technique and the second detectable moiety is detectable by a second imaging technique. A large variety of imaging agents with double detectability may thus be obtained. The simultaneous use of two different imaging agents (i.e., of a first imaging agent detectable by a first imaging technique and a second imaging agent detectable by a second imaging technique) is also contemplated.

The inventive imaging agents may be prepared by any synthetic method known in the art, the only requirement being that, after reaction, the hMMP-9 nucleic acid aptamer moiety and detectable moiety retain their affinity and detectability property, respectively. The hMMP-9 nucleic acid aptamer and detectable moieties may be associated in any of a large variety of ways. Association may be covalent or non-covalent. When the association is covalent, the hMMP-9 nucleic acid aptamer and detectable moieties may be bound to each other either directly or indirectly (e.g., through a linker). When the detectable moiety is a metal entity, the hMMP-9 nucleic acid aptamer moiety may be associated to the detectable metal entity via a metal-chelating moiety.

More specifically, in certain embodiments, the hMMP-9 nucleic acid aptamer moiety and detectable moiety are directly covalently linked to each other. The direct covalent binding can be through an amide, ester, carbon-carbon, disulfide, carbamate, ether, thioether, urea, amine or carbonate linkage. The covalent binding can be achieved by taking advantage of functional groups present on the hMMP-9 nucleic acid aptamer moiety and detectable moieties. Suitable functional groups that can be used to attach the two moieties together include, but are not limited to, amines (preferably primary amines), anhydrides, hydroxy groups, carboxy groups and thiols. A direct linkage may also be formed by using an activating agent, such as a carbodiimide, to bind, for example, the primary amino group present on one moiety to the carboxy group present on the other moiety. Activating agents suitable for use in the present invention are well known in the art.

In other embodiments, the hMMP-9 nucleic acid aptamer moiety and detectable moiety are indirectly covalently linked to each other via a linker group. This can be accomplished by using any number of stable bifunctional agents well known in the art, including homofunctional and heterofunctional linkers. The use of a bifunctional linker differs from the use of an activating agent in that the former results in a linking moiety being present in the inventive imaging agent after reaction, whereas the latter results in a direct coupling between the two moieties involved in the reaction. The main role of the bifunctional linker is to allow the reaction between two otherwise chemically inert moieties. However, the bifunctional linker, which becomes part of the reaction product, can also be selected such that it confers some degree of conformational flexibility to the imaging agent (e.g., the bifunctional linker may comprise a straight alkyl chain containing several atoms).

A wide range of suitable homofunctional and heterofunctional linkers known in the art can be used in the context of the present invention. Preferred linkers include, but are not limited to, alkyl and aryl groups, including straight chain and branched alkyl groups, substituted alkyl and aryl groups, heteroalkyl and heteroaryl groups, that have reactive chemical functionalities such as amino, anhydride, hydroxyl, carboxyl, carbonyl groups, and the like. Typically, a hexylamino linker may be used.

The association between the hMMP-9 nucleic acid aptamer moiety and the metal-chelating moiety is preferably covalent. Suitable metal-chelating moieties for use in the present invention may be any of a large number of metal chelators and metal complexing molecules known to bind detectable metal moieties. Preferably, metal-chelating moieties are stable, non-toxic entities that bind radionuclides or paramagnetic metal ions with high affinity.

Examples of metal-chelating moieties that have been used for the complexation of paramagnetic metal ions, such as gadolinium III (Gd³⁺), include S-acetylmercaptoacetyltriglycine (MAG3), DTPA (diethylene triaminepentaacetic acid); DOTA (1,4,7,10-tetraazacyclododecane-N,N′,N″,N′″-tetraacetic acid); and derivatives thereof (see, for example, U.S. Pat. Nos. 4,885,363; 5,087,440; 5,155,215; 5,188,816; 5,219,553; 5,262,532; and 5,358,704; and D. Meyer et al., Invest. Radiol. 1990, 25: S53-55), in particular, DTPA-bis(amide) derivatives (U.S. Pat. No. 4,687,659). Other metal-chelating moieties that complex paramagnetic metal ions include acyclic entities such as aminopolycarboxylic acids and phosphorus oxyacid analogues thereof (e.g., triethylenetetraminehexaacetic acid or TTHA), and dipyridoxal diphosphate (DPDP) and macrocyclic entities (e.g., 1,4,7,10-tetraazacyclododecane-N,N′,N″-triacetic acid or DO3A). Metal-chelating moieties may also be any of the entities described in U.S. Pat. Nos. 5,410,043; 5,277,895; and 6,150,376; or in F. H. Arnold, Biotechnol. 1991, 9: 151-156.

Examples of metal-chelating moieties that complex radionuclides, such as technetium-99m, include, for example, N₂S₂ and N₃S chelators (A. R. Fritzberg et al., J. Nucl. Med. 1982, 23: 592-598; U.S. Pat. Nos. 4,444,690; 4,670,545; 4,673,562; 4,897,255; 4,965,392; 4,980,147; 4,988,496; 5,021,556 and 5,075,099). Other suitable metal-chelating moieties can be selected from polyphosphates (e.g., ethylene diaminetetramethylenetetra-phosphonate, EDTMP); aminocarboxylic acids (e.g., EDTA, N-(2-hydroxyl)ethylene-diaminetriacetic acid, nitrilotriacetic acid, N,N-di(2-hydroxyethyl)glycine, ethylenebis(hydroxyphenylglycine) and diethylenetriamine pentacetic acid); 1,3-diketones (e.g., acetylacetone, trifluoroacetylacetone, and thenoyltrifluoroacetone); hydroxycarboxylic acids (e.g., tartaric acid, citric acid, gluconic acid, and 5-sulfosalicyclic acid); polyamines (e.g., ethylenediamine, diethylenetriamine, triethylenetetraamine, and triaminotriethylamine); aminoalcohols (e.g., triethanolamine and N-(2-hydroxyethyl)ethylenediamine); aromatic heterocyclic bases (e.g., 2,2′-diimidazole, picoline amine, dipicoline amine and 1,10-phenanthroline); phenols (e.g., salicylaldehyde, disulfopyrocatechol, and chromotropic acid); aminophenols (e.g., 8-hydroxyquinoline and oximesulfonic acid); oximes (e.g., hexamethylpropylene-amine oxime, HMPAO); Schiff bases (e.g., disalicylaldehyde 1,2-propylenediimine); tetrapyrroles (e.g., tetraphenylporphin and phthalocyanine); sulfur compounds (e.g., toluenedithiol, meso-2,3-dimercaptosuccinic acid, dimercaptopropanol, thioglycolic acid, potassium ethyl xanthate, sodium diethyldithiocarbamate, dithizone, diethyl dithiophosphoric acid, and thiourea); synthetic macrocyclic compounds (e.g., dibenzo[18]crown-6), or combinations of two or more of the above agents.

As can readily be appreciated by those skilled in the art, a hMMP-9-targeted imaging agent of the invention can comprise any number of hMMP-9 nucleic acid aptamer moieties and any number of detectable moieties, linked to one another by any number of different ways. The hMMP-9 nucleic acid aptamer moieties within an inventive imaging agent may be all identical or different. Similarly, the detectable moieties within an inventive imaging agent may be all identical or different. The precise design of a hMMP-9-targeted imaging agent will be influenced by its intended purpose(s) and the properties that are desirable in the particular context of its use.

In one embodiment, the nucleic acid aptamer of the invention consists of a nucleic acid sequence as set forth in SEQ ID NO:3 wherein the pyrimidines are 2′-fluoropyrimidine and the purine ribonucleosides are substituted by 2′O-methyl residues (F3Bomf) and which is conjugated at its 5′ end to S-acetylmercaptoacetyltriglycine (MAG3) through a hexylamino linker and labeled with ^{99m}Tc.

The invention provides reagents and strategies to image and detect the presence of hMMP-9. More specifically, the invention provides targeted reagents that are detectable by imaging techniques and methods allowing the detection, localization and/or quantification of hMMP-9 in in vitro and ex vivo systems as well as in living subjects, including human patients. The methods provided are based on the use of hMMP-9-targeted imaging agents comprising at least one hMMP-9 nucleic acid aptamer moiety having a high affinity and specificity for hMMP-9, associated with at least one detectable moiety that allows visualization of the imaging agent using imaging techniques.

More specifically, the present invention provides methods for detecting the presence of hMMP-9 in a biological system comprising the step of contacting the biological system with an effective amount of a hMMP-9-targeted imaging agent of the invention, or a pharmaceutical composition thereof. The contacting is preferably carried out under conditions that allow the imaging agent to interact with hMMP-9 present in the system so that the interaction results in the binding of the imaging agent to the hMMP-9. The imaging agent that is bound to hMMP-9 present in the system is then detected using an imaging technique. One or more images of at least part of the biological system may be generated. The contacting may be carried out by any suitable method known in the art. For example, the contacting may be carried out by incubation.

The biological system may be any biological entity that can produce and/or contain hMMP-9. For example, the biological system may be a cell, a biological fluid or a biological tissue. The biological system may originate from a living subject (e.g., it may be obtained by drawing blood, by biopsy or during surgery) or a deceased subject (e.g., it may be obtained at autopsy).

The subject is a patient suspected of having a clinical condition associated with hMMP-9.

The present invention also provides methods for detecting the presence of hMMP-9 in a patient. The methods comprise administering to the patient an effective amount of a hMMP-9-targeted imaging agent of the invention, or a pharmaceutical composition thereof. The administration is preferably carried out under conditions that allow the imaging agent (1) to reach the area(s) of the patient's body that may contain abnormal hMMP-9 (i.e., hMMP-9 associated with a clinical condition) and (2) to interact with such hMMP-9 so that the interaction results in the binding of the imaging agent to the hMMP-9. After administration of the hMMP-9-targeted imaging agent and after sufficient time has elapsed for the interaction to take place, the imaging agent bound to abnormal hMMP-9 present in the patient is detected by an imaging technique. One or more (e.g., a series) images of at least part of the body of the patient may be generated. One skilled in the art will know, or will know how to determine, the most suitable moment in time to acquire images following administration of the imaging agent. Depending on the imaging technique used (e.g., MRI), one skilled in the art will also know, or know how to determine, the optimal image acquisition time (i.e., the period of time required to collect the image data).

Administration of the hMMP-9-targeted imaging agent, or pharmaceutical composition thereof, can be carried out using any suitable method known in the art such as administration by oral and parenteral methods, including intravenous, intraarterial, intrathecal, intradermal and intracavitory administrations, and enteral methods.

As mentioned above, the imaging agent bound to hMMP-9 (present either in a biological system or in a patient) is detected using an imaging technique such as contrast-enhanced ultrasonography, planar scintigraphy, SPECT, MRI, fluorescence spectroscopy, or a combination thereof.

The methods of the invention that provide for detecting the presence of hMMP-9 in a patient or in a biological system obtained from a patient can be used to diagnose a pathological condition associated with hMMP-9. The diagnosis can be achieved by examining and imaging parts of or the whole body of the patient or by examining and imaging a biological system (such as one or more samples of biological fluid or biological tissue) obtained from the patient. One or the other method, or a combination of both, will be selected depending of the clinical condition suspected to affect the patient. Comparison of the results obtained from the patient with data from studies of clinically healthy individuals will allow determination and confirmation of the diagnosis.

These methods can also be used to follow the progression of a pathological condition associated with hMMP-9. For example, this can be achieved by repeating the method over a period of time in order to establish a time course for the presence, localization, distribution, and quantification of “abnormal” hMMP-9 in a patient.

These methods can also be used to monitor the response of a patient to a treatment for a pathological condition associated with hMMP-9. For example, an image of part of the patient's body that contains “abnormal” hMMP-9 (or an image of part of a biological system originating from the patient and containing “abnormal” hMMP-9) is generated before and after submitting the patient to a treatment. Comparison of the “before” and “after” images allows the response of the patient to that particular treatment to be monitored.

Pathological conditions that may be diagnosed, or whose progression can be followed using the inventive methods provided herein may be any disease and disorder known to be associated with hMMP-9, i.e., any condition that is characterized by undesirable or abnormal interactions mediated by hMMP-9. Examples of such conditions include but are not limited to inflammation, neurovascular and neurodegenerative diseases (such as brain injury, stroke, or hemorrhagic transformation), atherosclerosis and cancers.

In one embodiment the patient suffers from a cancer selected from the group consisting of adrenal cortical cancer, anal cancer, bile duct cancer (e.g. periphilar cancer, distal bile duct cancer, intrahepatic bile duct cancer), bladder cancer, bone cancer (e.g. osteoblastoma, osteochondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating lobular carcinoma, lobular carcinoma in situ, gynecomastia), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adenocarcinoma, clear cell), esophagus cancer, gallbladder cancer (mucinous adenocarcinoma, small cell carcinoma), gastrointestinal carcinoid tumors (e.g. choriocarcinoma, chorioadenoma destruens), Hodgkin's disease, non-Hodgkin's lymphoma, Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma, thyroid lymphoma), vaginal cancer, vulvar cancer, and uterine cancer (e.g. uterine leiomyosarcoma). Generally, the cancer is characterized by the presence of at least one solid tumor.

In the methods of detection/imaging of hMMP-9 and of diagnosis of pathological conditions associated with hMMP-9 described herein, the imaging agents of the present invention may be used per se or as a pharmaceutical composition. Accordingly, in one aspect, the present invention provides pharmaceutical compositions comprising at least one hMMP-9 nucleic acid aptamer of the invention. In a related aspect, the present invention provides pharmaceutical compositions comprising at least one hMMP-9-targeted imaging agent of the invention (or any physiologically tolerable salt thereof), and at least one pharmaceutically acceptable carrier.

The specific formulation will depend upon the selected route of administration. Depending on the particular type of pathological condition suspected to affect the patient and the body site to be examined, the imaging agent may be administered locally or systemically, delivered orally (as solids, solutions or suspensions) or by injection (for example, intravenously, intraarterially, intrathecally (i.e., via the spinal fluid), intradermally or intracavitory).

Often, pharmaceutical compositions will be administered by injection. For administration by injection, pharmaceutical compositions of imaging agents may be formulated as sterile aqueous or non-aqueous solutions or alternatively as sterile powders for the extemporaneous preparation of sterile injectable solutions. Such pharmaceutical compositions should be stable under the conditions of manufacture and storage, and must be preserved against the contaminating action of microorganisms such as bacteria and fungi.

Pharmaceutically acceptable carriers for administration by injection are solvents or dispersion media such as aqueous solutions (e.g., Hank's solution, alcoholic/aqueous solutions, or saline solutions) and non-aqueous carriers (e.g., propylene glycol, polyethylene glycol, vegetable oil and injectable organic esters such as ethyl oleate). Injectable pharmaceutical compositions may also contain parenteral vehicles (such as sodium chloride and Ringer's dextrose), and/or intravenous vehicles (such as fluid and nutrient replenishers); as well as other conventional, pharmaceutically acceptable, non-toxic excipients and additives including salts, buffers, and preservatives such as antibacterial and antifungal agents (e.g., parabens, chlorobutanol, phenol, sorbic acid, thimerosal, and the like). Prolonged absorption of the injectable compositions can be brought about by adding agents that can delay absorption (e.g., aluminum monostearate and gelatin). The pH and concentration of the various components can readily be determined by those skilled in the art.

Sterile injectable solutions are prepared by incorporating the active compound(s) and other ingredients in the required amount of an appropriate solvent, and then by sterilizing the resulting mixture, for example, by filtration or irradiation. The methods of manufacture of sterile powders for the preparation of sterile injectable solutions include vacuum drying and freeze-drying techniques.

In general, the dosage of the hMMP-9 nucleic acid aptamer of the invention or a hMMP-9-targeted imaging agent (or pharmaceutical composition thereof) will vary depending on considerations such as age, sex and weight of the patient, as well as the particular pathological condition suspected to affect the patient, the extent of the disease, the area(s) of the body to be examined, and the sensitivity of the detectable moiety. Factors such as contraindications, therapies, and other variables are also to be taken into account to adjust the dosage of imaging agent to be administered. This, however, can be readily achieved by a trained physician.

In general, a suitable daily dose of a hMMP-9-targeted imaging agent (or pharmaceutical composition thereof) corresponds to the lowest amount of imaging agent (or pharmaceutical composition) that is sufficient to allow detection/imaging of any relevant (i.e., generally overexpressed) hMMP-9 present in the patient. To minimize this dose, it is preferred that administration be intravenous, intramuscular, intraperitoneal or subcutaneous, and preferably proximal to the site to be examined. For example, intravenous administration is appropriate for imaging the cardio/neurovascular system; while intraspinal administration is better suited for imaging of the brain and central nervous system.

In another aspect, the present invention provides kits comprising materials useful for carrying out the diagnostic methods of the invention. The diagnostic procedures described herein may be performed by clinical laboratories, experimental laboratories, or practitioners.

In certain embodiments, an inventive kit comprises at least one hMMP-9 nucleic acid aptamer and at least one detectable entity, and, optionally, instructions for associating the hMMP-9 nucleic acid aptamer and detectable entity to form a hMMP-9-targeted imaging agent according to the invention. The detectable entity is preferably a short-lived radionuclide such as technetium-99m (^{99m}Tc), gallium-67 (⁶⁷Ga), yttrium-91 (⁹¹Y), indium-111 cm), rhenium-186 (¹⁸⁶Re) and thallium-201 (²⁰¹Tl). Preferably, the hMMP-9 nucleic acid aptamer and detectable entity are present, in the kit, in amounts that are sufficient to prepare a quantity of imaging agent that is suitable for the detection of hMMP-9 and diagnosis of a particular clinical condition in a subject.

In other embodiments, an inventive kit comprises at least one hMMP-9-targeted imaging agent according to the invention. In such embodiments, the hMMP-9-targeted imaging agent is preferably chemically stable.

A kit according to the present invention may further comprise one or more of: labeling buffer and/or reagent; purification buffer, reagent and/or means; injection medium and/or reagents. Protocols for using these buffers, reagents and means for performing different steps of the preparation procedure and/or administration may be included in the kit.

The different components included in an inventive kit may be supplied in a solid (e.g., lyophilized) or liquid form. The kits of the present invention may optionally comprise different containers (e.g., vial, ampoule, test tube, flask or bottle) for each individual component. Each component will generally be suitable as aliquoted in its respective container or provided in a concentrated form. Other containers suitable for conducting certain steps of the preparation methods may also be provided. The individual containers of the kit are preferably maintained in close confinement for commercial sale.

In certain embodiments, a kit further comprises instructions for using its components for the diagnosis of clinical conditions associated with hMMP-9 according to a method of the present invention. Instructions for using the kit according to a method of the invention may comprise instructions for preparing an imaging agent from the hMMP-9 nucleic acid aptamer and detectable moiety, instructions concerning dosage and mode of administration of the imaging agent, instructions for performing the detection of hMMP-9, and/or instructions for interpreting the results obtained. A kit may also contain a notice in the form prescribed by a governmental agency regulating the manufacture, use or sale of pharmaceuticals or biological products.

The invention will be further illustrated by the following figures and examples. However, these examples and figures should not be interpreted in any way as limiting the scope of the present invention.

### FIGURES

FIG. 1: Secondary structure prediction of aptamers F1, F2, F3 selected against hMMP-9 protein and of the truncated variant F3B. G-U pairs have been taken into account. Nucleotides 1-19 and 50-68 correspond to fixed flanks of the candidate sequences.

FIG. S1: Secondary structure of F3 hMMP-9 aptamer variants. Predicted structures of truncated F3 variants: A) F3B (the shortened variant), F3C1 (deletion of the two terminal base pairs of F3B), F3C2 (deletion of the first internal loop of F3B), F3BA (ten A apical loop), F3BΔ (apical loop substituted by a hexaethylene glycol linker), B) F3BdS (internal loop with six abasic sites), F3BP (internal loop with six A residues), F3BAA (C,C mismatch of F3B replaced by A,A mismatch), F3BCG (C,C mismatch of F3B replaced by a CG pair), F3Binv1 (stem strands exchanged in the upper part of the stem) and F3Binv2 (stem strands exchanged in the lower part of the stem).

### EXAMPLE 1

### A 99mTc-MAG3-Aptamer for Imaging Human Tumours Associated with High Level of Matrix Metalloproteinase-9

Materials. Aptamer Production and Analysis

SELEX Conditions

The oligonucleotide library was obtained by transcription from a DNA library, synthesized by Proligo, containing 30 random nucleotides (N30) flanked by invariant primer annealing sites: 5′-GTGTGACCGACCGTGGTGC-N30-GCAGTGAAGGCTGGTAACC-3′. (SEQ ID NO:9). Two different primers P20 5′GTGTGACCGACCGTGGTGC (SEQ ID NO:10) and 3′ SL 5′TAATACGACTCACTATAGGTTACCAGCCTTCACTGC (SEQ ID NO:11) containing the T7 transcription promoter (underlined), were used for PCR amplification. The modified 2′-fluoropyrimidine RNA library used for the selection and aptamer F3 were obtained by transcription (DuraScribe T7 transcription kit from Epicentre Technologies containing 2′-F-CTP and 2′-F-UTP). The mutant T7 RNA polymerase Y639F (38) was also used.

The in vitro selection against hMMP-9 protein (Calbiochem) was performed at 23° C. in SP buffer (50 mM Tris HCl, pH 7.4, 50 mM NaCl, 100 mM KCl, 5 mM CaCl₂, 1 mM magnesium acetate) using the filter retention technique (HAWP 0.45 μM, Millipore) (39). Filters were pretreated with alkali as described by McEntee et al. (40) in order to reduce non-specific adsorption of nucleic acids. Library was first incubated with the alkali-treated filters during 20 min then with hMMP-9 protein for 20 min. The mixture was filtered and filters washed with SP buffer. Candidates bound to the protein were eluted with 500 μl phenol/urea 7M for 20 min at 65° C. and reverse transcribed with 200 U M-MLV reverse transcriptase RNase if Point Mutant (Promega). 1 μl of which was used for 25 cycles of PCR at 63° C. with 1 U of AmpliTaq Gold DNA polymerase (Applied Biosystems) and the two P20 and 3′SL primers. 2′-F-RNA candidates were obtained by in vitro transcription of the PCR products with the DuraScribe T7 transcription kit (Epicentre Technologies). During the successive in vitro selection rounds, candidates and protein concentrations were progressively decreased whereas the number of washes, used to eliminate weak binders, was increased. This resulted in a tougher competition between the candidates for binding as evolution proceeded. Monitoring the evolution of the binding properties of the selected population after every cycle indicated an increase in binding efficiency of the candidates up to round 15. After 14 selection cycles against the hMMP-9 protein, selected candidates were cloned using the TOPO TA cloning kit (Invitrogen) and sequenced (Genome Express Company).

Oligonucleotides Synthesis

The truncated variants F3B, F3C1 and F3C2 of the aptamer F3, the 2′O-methyl purine/2′Fluoro pyrimidine F3B (F3Bomf) derivatives, the 3′end biotinylated aptamers (F3B, F3Bomf), the control sequence 5′UGCCAAACGCGUCCCCUUUGCCCGGCCUCCGCCGCA 3′ (SEQ ID NO:12) and the mutants F3BΔ, F3BA were chemically synthesized on an Expedite 8909 in our laboratory according to standard procedures. All oligonucleotides were purified by electrophoresis on denaturing 20% polyacrylamide, 7 M urea gels. Secondary structure prediction of aptamers was determined using the mfold web server (http://mfold.rna.albany.edu/?q=mfold).

NMR Analysis of F3B Variant

1H NMR spectra were recorded at pH 6.4 and 5.5, in 10 mM sodium phosphate buffer containing 90/10 H2O/D2O. Imino protons were assigned based on the analysis of NOESY spectra recorded at 4° C. and 15° C.

Oligonucleotide Conjugation

Oligonucleotides F3Bomf and the control sequence, bearing a 5′ hexylamino function, were synthesized on a 1 micromole scale with an ABI Expedite 8909 synthesizer, using conventional β-cyanoethyl phosphoramidite chemistry. Once purified (HPLC, Macherey-Nagel Nucleodur® column, 0.1 M triethylammonium acetate, pH 7.0, (acetonitrile/0.1 M triethylammonium acetate, pH 7.0: 80/20) gradient), they were conjugated to MAG3 (41). Briefly, 20 nmol of oligonucleotide were suspended in 100 μl of binding buffer (sodium bicarbonate/sodium carbonate 0.25 M, pH 8.3, sodium chloride 1 M, sodium ethylenediaminetetraacetate 1 mM), and gently stirred at room temperature. MAG3-NHS (3 mg, in 30 μl of DMF) was added in portions at room temperature over 3 h. After complete addition, the suspension was stirred for an additional hour, and the crude was directly purified by HPLC under the same conditions to afford the oligonucleotide-MAG3 conjugate in 50-90% yield. Conjugates characterization was performed with a MALDI-ToF mass spectrometer (Reflex III, Bruker).

Human Matrix Metalloprotease-9

The human MMP-9 was purchased to Calbiochem; samples were checked for purity by SDS polyacrylamide gel electrophoresis. Batch to batch variation was noticed resulting in the presence of breakdown fragments likely related to self-cleavage of the protease. Only samples with low fragment content were used in our study.

Binding and Specificity Assays

The dissociation constant (Kd) of the complexes, formed by the aptamers and the hMMP-9 protein, was determined using the filter retention method. 1 nM of ³²P 5′end-labeled aptamer was incubated with increasing concentrations of hMMP-9 (10, 20, 40, 72, 160, 320, 500 nM) for 20 min at 23° C. in 20 μl SP buffer. Complexes were filtered and the radioactivity retained on the filter was quantified using a scintillation counter (LS 6000 IC, Beckman). Kd values were deduced from data point fitting with Kaleidagraph 3.0 (Abelbeck software), according to the equation: B=(Bmax[L]₀)/(H₀+Kd), where B is the proportion of complex, Bmax is the maximum of complex formed and [L]₀ is the total concentration of unlabeled ligand.

Surface Plasmon Resonance (SPR) experiments were performed on a BIAcore™ 3000 apparatus (Biacore AB, Sweden). 2 μg of hMMP-9 protein were injected on a carboxymethylated dextran CM5 sensorchip for immobilisation and aptamers F3B, F3C1 and F3C2 were injected at 200 nM (20 μl/min) in SP buffer. Alternatively CM5 sensorchips were functionalized with streptavidin. 3′ end biotinylated F3B and F3Bomf were immobilised on the functionalized CM5 and hMMP-9 protein at 100 nM in SP buffer was injected. hMMP-9 was injected at different concentrations (from 5 to 160 nM) in PBS buffer. In another series of experiments 3′end biotinylated aptamer F3Bomf was immobilized and hMMP-9, pro-hMMP-9, mouse pro-MMP-9, human MMP-2 or MMP-7 proteins were injected at 50 nM in PBS buffer. SPR experiments were performed at 23° C., at 20 μl/min, and the complexes were dissociated with a pulse of a solution containing 40% formamide/3.6 M urea/30 mM EDTA (42).

^{99m}Tc Oligonucleotides Radio Labelling

The MAG3 F3Bomf and the control sequence were labelled with ^{99m}Tc as described by Winnard et al. (41): two fresh solutions were prepared: i) sodium tartrate (50 mg/ml) in sterile 0.5 M sodium bicarbonate, 0.25 M ammonium acetate, 0.18 M ammonium hydroxide, pH 9.2. (The high pH of the tartrate solution was necessary so that the final pH is approximately 7.6) and ii) a 1 mg/ml SnCl₂.2H₂O in 10 mM hydrochloric acid just prior to use. ^{99m}Tc pertechnetate solution (2-10 μl) (Elumatic III—Cis Bio International) was added to the MAG3-aptamer (10-100 μg) to provide about 3.7 MBq/μg of aptamer followed by the addition of the tartrate solution to a final concentration of 6-7 μg/μl. The stannous ion solution was added immediately thereafter (1 μg of SnCl₂.2H₂O for each 10 μg of aptamer) and left at room temperature for 15 min. The labelled aptamer was then purified by micro spin column (MicroSpin G-25 columns, GE Healthcare) and the radiochemical purity was determined using thin-layer chromatography (TLC) (TLC plates RP-18, Merck).

Under the above set of conditions, average labelling efficiency of 70% (N=15, SD=14%) was achieved. Radiochemical purity (RCP) determined by thin layer chromatography (TLC) was 77% (SD=8%). The stability of ^{99m}Tc-MAG3-aptamer over time was determined using TLC. The radiolabelled oligonucleotides RCP was about 70% at 6 h following radiolabelling, indicating good stability. Average specific activities of 2.48 MBq/μg were obtained (SD=10%).

Tissue Samples and In Vitro Binding Assay

Tumour tissues used in these studies were obtained from the department of Pathology, University Hospital Bordeaux, France. Nine different types of well characterized tumours: pilocytic astrocytoma grade 1, meningioma grade 1, fibrillary astrocytoma grade 2, ependymoma grade 2, anaplastic astrocytoma grade 3, medulloblastoma grade 4, primitive central nervous system lymphoma grade 4, glioblastoma grade 4 were collected from surgical samples, including one case of normal brain tissue from a patient undergoing autopsy. Tumour grade was done according to the 2007 WHO classification of tumours of the central nervous system (1). All tissue samples were formalin-fixed and paraffin-embedded. Representative 2.5 μm-thick tissue sections were obtained from blocks of paraffin-embedded tissue and subjected to immunohistochemistry and autoradiography analysis.

Binding studies were performed using these tissue sections incubated in the presence of either ^{99m}Tc-MAG3-F3Bomf aptamer or ^{99m}Tc-MAG3-control sequence according to the following procedure: after deparaffinization and rehydration, tissue slices were incubated with 0.037 MBq (0.00125 nmol) of ^{99m}Tc-MAG3-F3Bomf and adjacent section with ^{99m}Tc-MAG3-control for one hour in a humidified chamber at room temperature before being washed twice in PBS+0.1% Tween and then twice in purified water. Then sections were imaged using a Beta Imager 2000 (Biospace Mesures, Paris, France).

Immunostaining with hMMP-9 Antibodies

Immunohistochemical hMMP-9 detection was performed on serial 2.5 μm-thick sections, using a purified anti-mouse hMMP-9 monoclonal antibody (ab58803, Abcam). Immunohistochemical procedures were carried out with a DAKO Envision Peroxidase System (DAKO Diagnostica) according to the following protocol: paraffin-embedded sections were deparaffinized with xylene, dehydrated through a graded alcohol series and washed with distilled water. They were then treated with 0.3% hydrogen peroxydase for 5 minutes to block endogenous peroxidase activity. After washing with PBS, the slides were incubated for 30 minutes with hMMP-9 antibody diluted 1:150 in a humidified chamber at room temperature and then washed twice in PBS. En Vision multi-link was then applied as the secondary antibody for 30 min before washing and incubation with diaminobenzydine DAB substrate for 10 min, and hematoxylin counterstaining Appropriate positive and negative controls omitting the primary antibody were included with every slide run. Immunoreactivity was evaluated in the cell cytoplasm, cytoplasmic membrane and in the extra-cellular matrix.

Results:

Characteristics and Binding Properties of hMMP-9 Aptamers

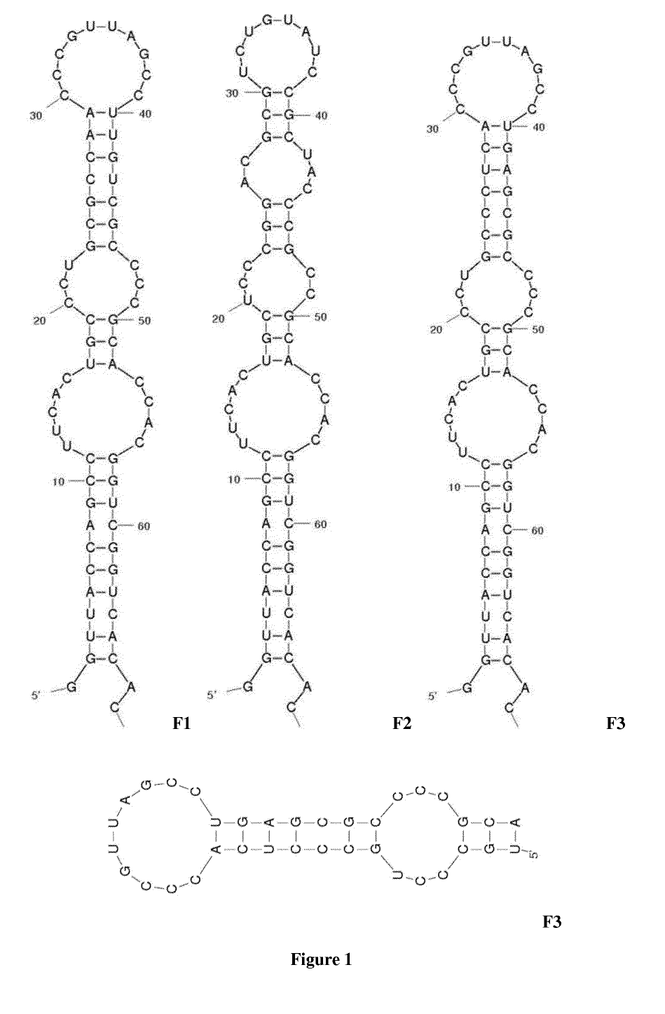

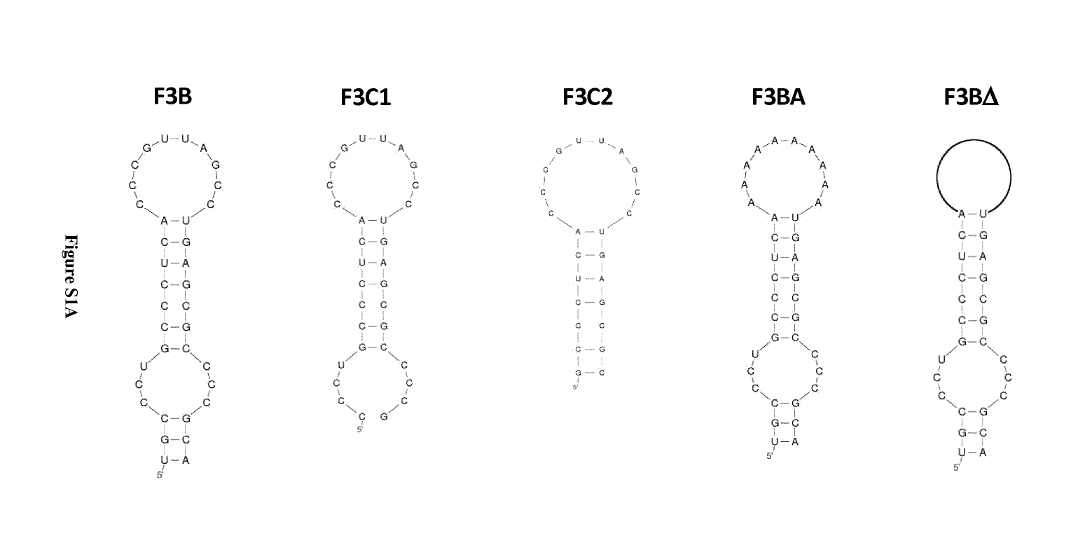

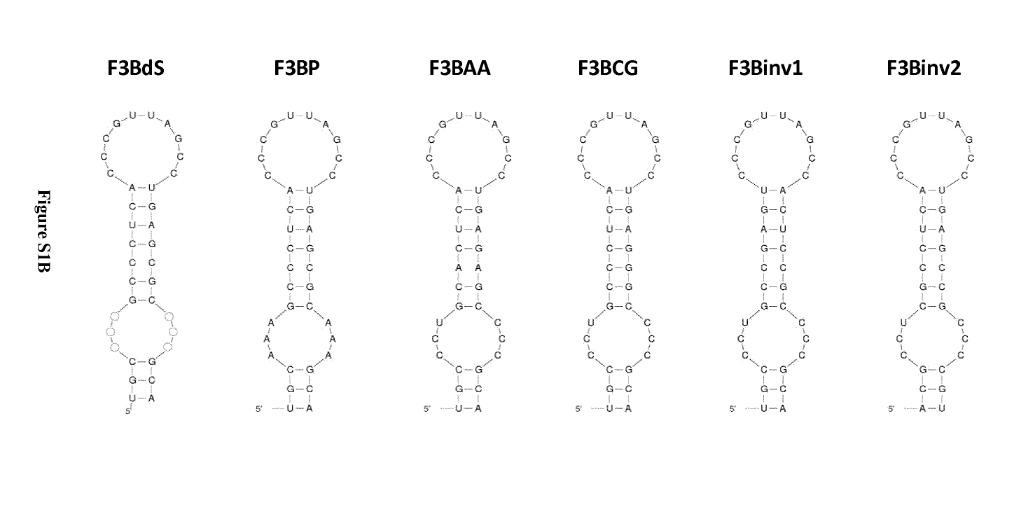

The SELEX strategy has been carried out against the recombinant human MMP-9 protein (gelatinase B), using a library of RNA candidates containing 2′fluoropyrimidine nucleoside residues, as described in Material and Methods. The 30 nt random window is flanked by fixed regions that display partial complementarity, generating hairpin-like candidates through the formation of a weak duplex between the 5′ and 3′ parts. This is expected to limit the contribution of the fixed parts to the interaction of the selected aptamer with the target and to some extent pre-organize the candidates as hairpins. After 15 selection rounds, the binding properties of the selected populations improved as monitored by SPR analysis (not shown): when flowing the successive pools on a hMMP-9-grafted biochip, the resonance signal increased for rounds 9^{th} to 14^{th} and decreased markedly for the 15^{th} round. 77 clones were sequenced from the 14^{th} round of selection and compared. Three sequences named F1, F2 or F3 represented 74% of the candidates. Secondary structure prediction of the three sequences using mfold, showed a very high degree of similarity; selected candidates appeared as imperfect hairpins (FIG. 1). The bottom part of F1, F2 and F3 adopts a mismatched double-stranded structure contributed by the fixed regions. The folding of what corresponds to the random region of the library shows from bottom to top: a 5 or 6 nt long pyrimidine rich internal loop and a 6 base pair G-C rich stem interrupted by a mismatch (F1 and F3) or a 5 nt internal loop (F2). F1 and F3 are predicted to form the same 10 nt long apical loop whereas the F2 one is only 8 nt in length; both loops are pyrimidine rich (FIG. 1). F1 and F3 display about 80% sequence homology in the 30 nt random region.

Binding curves of ³²P 5′ end-labelled F1, F2 and F3 aptamers to hMMP-9 were determined by filter retention assay revealing a similar affinity of F1, F2 or F3 for hMMP-9. Aptamer F3, with an equilibrium dissociation constant of 8.1±3.4 nM was a slightly stronger hMMP-9 binder than F2 (Kd=15.4±2.8 nM) or F1 (Kd=18.3±3.7 nM) and was chosen for further investigations.

MMPs constitute a large family of closely related enzymes. In order to assess the specificity of the aptamer F3 for hMMP-9, we monitored its binding efficiency to the human MMP-2 (hMMP-2), the matrix metalloprotease closest to hMMP-9 also called gelatinase A and to the human MMP-7 (hMMP-7) by the filter retention procedure. Binding of F3 to hMMP-9 and pro-hMMP-9 was specific: no retention was detected by either hMMP-7 or its proform whereas a light signal was noticed with hMMP-2.

In order to make the synthesis and the study easier, we undertook the truncation of F3 down to the minimal size compatible with hMMP-9 binding. On the basis of the predicted structure F3 was shortened from 68 to 36 nucleotides (variant F3B; FIG. 1), thus getting rid of most of the primer sequences but 3 residues on each side of the bottom stem. The ¹H NMR analysis of the resulting F3B showed a spectrum of exchangeable protons consistent with the hairpin structure shown on FIG. 1A retaining a C,C mismatch in the upper part of the stem and a pyrimidine internal hexaloop in the bottom part, under the conditions of the experiment (not shown). F3B was further shortened thus generating two variants: F3C1 (nt 3-34 of F3B, deletion of the two first base pairs of F3B; predicted abolition of the bottom double-stranded stem) and F3C2 (nt 7-30 of F3B, deletion of the first internal loop of F3B) (FIG. S1). BIAcore analysis demonstrated that F3B showed the best binding efficiency for hMMP-9 whereas F3C1 was a weaker binder and F3C2 hardly yielded a SPR signal. This suggests that the bottom internal loop of the aptamer is essential for the interaction with hMMP-9.

We then synthesised a number of mutated F3B derivatives in order to delineate the structural elements of the aptamer contributing to its binding to the target protein. Loop regions are generally crucial for the formation of aptamer-target complexes. The importance of the apical loop was firstly investigated. In F3BΔ the nt 14-23 of F3B were substituted by a hexaethylene glycol linker whereas the variant F3BA displayed a A₁₀ loop (FIG. S1). This resulted in a tremendously decreased (F3BA) or abolished (F3BΔ) interaction with hMMP-9 (not shown). We then modified the composition of the internal loop. This region originally composed of pyrimidine residues was replaced by six abasic sites (F3Bds) or replaced by 6 A residues (F3BP) (FIG. S1). We also noticed a drastically decreased binding signal for either derivative (not shown). Finally, F3B stem was also modified. First, the C(9),C(28) mismatch in the upper stem was replaced either by an A,A mismatch, allowing the conservation of the secondary structure or by a CG pair, allowing formation of a perfect double-stranded stem (FIG. S1). Both variants bound to hMMP-9 with an efficiency similar to the parent F3B indicating that the C,C mismatch was not essential for the interaction with hMMP-9. Indeed this element was not conserved in F1 or F2 even though these aptamers did not display a perfect double-stranded upper stem either. Second, the strands of the stems of F3B were exchanged, the original 5′ strand being placed on the 3′ side and vice versa, leading to a preserved secondary structure (FIG. S1). This did not alter the binding between hMMP-9 and the derived aptamer. We conclude that apical and internal loops were the two major F3B elements ensuring the formation of the F3B/hMMP-9 complex.

Next we optimized the F3B chemistry in the perspective of its use in biological media. In order to supply a fully nuclease resistant molecular tool, the original purine ribonucleosides were substituted by 2′O-methyl residues. The resulting aptamer F3Bomf was still able to interact with hMMP-9 with a similar efficiency as the parent F3B as indicated by SPR signal (750 RU and 805 RU under the conditions of our experiment, respectively). Both sensorgrams have close profiles, with a slower dissociation phase observed for the parent F3B. The binding of this F3Bomf derivative to hMMP-9 is specific: a control scrambled sequence with the same length, same base composition and same chemistry did not lead to a detectable SPR signal. Sensorgrams for the complex F3Bomf/hMMP-9 carried out at different protein concentrations could not be properly fitted to a 1:1 model preventing the accurate determination of kₒₙ and k_{off}. However the equilibrium constant was evaluated to be in the low nanomolar range. This aptamer bound to pro-hMMP-9 and processed hMMP-9 with a similar efficiency but with different binding and dissociation behaviour suggesting that it likely recognizes a site exposed in both active and inactive forms of the protein. In contrast, F3Bomf was able to discriminate between the human and the murine zymogen pro-MMP-9: a weak signal (49 RU) to the mouse pro-MMP-9 was detected, compared to about 1 300 RU with the human proenzyme. The specificity of the parent aptamer was also maintained following modification as F3Bomf did not bind to either hMMP-7 or hMMP-2. Surprisingly, the fully modified 2′O-methylribo aptamer does not allow the formation of a complex with hMMP-9.

The modified aptamer F3Bomf was then functionalized by conjugation at its 5′ end to S-acetylmercaptoacetyltriglycine (MAG3) through a hexylamino linker. This modification did not interfere with target recognition; the MAG3-F3Bomf aptamer did bind with hMMP-9 whereas pre-incubation of the protein with the functionalized aptamer abolished the SPR signal. Both the aptamer and the control oligonucleotide were then labelled with ^{99m}Tc as described in Materials and Methods for imaging hMMP-9 in tissues.

Human Central Nervous System Tumor Imaging

MMP-9 expression in several human tumours from central nervous system was investigated either by immunohistochemistry using specific hMMP-9 antibody or by binding assay with ^{99m}Tc-MAG3-F3Bomf anti-hMMP-9 aptamer. Immunohistochemical analysis revealed that hMMP-9 was highly expressed in glioblastomas. Strong cytoplasmic reactivity was observed for numerous tumour cells. Immunopositivity was also present in the extracellular matrix, as well as in the endothelial cells of blood vessels in the tumour environment. Of note the antibody used for this experiment was raised against the mouse MMP-9 that does not discriminate between murine and human enzymes in contrast to the aptamer F3Bomf. Therefore the aptamer shows a higher degree of specificity than the antibody. Incubation of glioblastoma slices adjacent to the ones used for immunohistochemical analysis with the radiolabelled aptamer F3Bomf, revealed a strong signal whereas the radiolabelled control sequence induced a weaker signal. Incubation with ^{99m}Tc-MAG3 alone did not produce any detectable signal. Pre-saturation of the slice with unlabelled MAG3-F3Bomf almost abolishes the radiolabelling by the ^{99m}Tc-aptamer whereas pre-saturation with unlabelled MAG3-control sequence did not prevent the labelling with ^{99m}Tc-MAG3-F3Bomf. This indicates that firstly the aptamer is able to bind to its target in the environment of the tumor and secondly that its binding to hMMP-9 is specific.

A range of other human central nervous system (CNS) tumour types also express MMP-9 (43-46). We investigated the imaging properties of the anti-hMMP-9 aptamer against pilocytic astrocytoma, meningioma, fibrillary astrocytoma, ependymoma, anaplastic astrocytoma, medulloblastoma, lymphoma and glioblastoma. The hMMP-9 expression, monitored by the immunohistochemical method revealed a cytoplasmic staining dependent on the tumour grade within the group of glial infiltrative tumours. For the other primitive brain tumours explored (pilocytic astrocytoma grade 1, fibrillary astrocytoma grade 2, anaplastic astrocytoma grade 3, glioblastoma grade 4, ependymoma grade 2, meningioma grade1 and medulloblastoma grade 4), hMMP-9 immunostaining showed a variable intensity of cytoplasmic expression. In all cases (glial and other tumour type), immunostaining for hMMP-9 was also clearly observed both within extra-cellular environment and endothelial cells. For primitive central nervous system lymphoma, hMMP-9 expression was weak in cytoplasmic compartment and in extra-cellular matrix. Healthy brain was used as control: no immunoreactivity for hMMP-9 was detected. The same tumors were incubated with the labelled anti-hMMP-9 aptamer. Generally, ^{99m}Tc-MAG3-F3Bomf induced a strong signal on the tissues whereas a much weaker signal was recorded with the control sequence. No signal was detected with healthy brain tissue. Therefore whatever the tumour type a perfect agreement was observed between antibody fixation and radiolabelled F3Bomf aptamer binding.

### CONCLUSION

Aptamers are attractive in biomedicine because of their advantages over antibodies which rely for instance on their reproducible chemical production, low immunogenicity, reversible denaturation and small size. Aptamers show many of the requested criteria for the ideal imaging probe. They are high affinity binding ligands, show high tissue-specific retention and rapid blood clearance for in vivo imaging (18). Because of their easy conjugation to the appropriate label (fluorophore, radionuclide), aptamers afford a valuable alternative to antibodies for protein detection.

MMPs are relevant marker of tumor malignancy. So far, molecular imaging of MMPs has been performed in tumor-bearing mice with fluorescent peptide substrates (47) or in human carotid (48) using MMP inhibitor radiotracers (49-50) or proteolytic nanobeacon (51). Investigation of MMP-2 and hMMP-9 expression with a ⁶⁴Cu radiolabeled cyclic peptide by microPET failed to demonstrate a specific uptake in gelatinase-expressing tumors (52) whereas the same cyclic peptide ⁶⁸Ga-DOTA conjugated shown acceptable plasma stability and good visualization of tumor xenografts (53). Recently, a ^{99m}Tc-monoclonal antibody was developed to target a membrane MMP for imaging atherosclerosis (54).

In this work, we have successfully obtained and characterized aptamers displaying high affinity for hMMP-9 protein. Aptamer F3 was shortened and modified to generate MAG3-F3Bomf, an aptamer-based imaging probe. We could detect specifically hMMP-9 protein, a tumor biomarker, on different human tumor slices with ^{99m}Tc-MAG3-F3Bomf. It is the first aptamer application for hMMP-9 detection. Its high specificity will improve the signal to noise ratio compared to broad-spectrum MMP inhibitors which lead to high uptake in tissues with non pathologic MMP expression.

Our goal is to develop an aptamer-based imaging tool for specific tumor monitoring in clinical studies. Chemistry and size have been optimized but future improvements may also be scheduled in order to enhance its retention in vivo (i.e multimers, pegylation). Aptamer injections in human tumor-bearing mice are scheduled.

### REFERENCES

Throughout this application, various references describe the state of the art to which this invention pertains. The disclosures of these references are hereby incorporated by reference into the present disclosure.
1. Louis, D. N., Ohgaki, H., Wiestler, O. D. and Cavenee, W. K. (eds.) (2007) WHO classification of tumours of the central nervous system. Fourth Edition IARC. WHO ed.
2. Peter, C., Burger, M. D., Bernd, W. and Scheithauer, M. D. (eds.) (2007) Tumors of the Central Nervous System. AFIP ed. ARP.
3. Laperriere, N., Zuraw, L. and Cairncross, G. (2002) Radiotherapy for newly diagnosed malignant glioma in adults: a systematic review. Radiother Oncol, 64, 259-273.
4. Heimberger, A. B., McGary, E. C., Suki, D., Ruiz, M., Wang, H., Fuller, G. N. and Bar-Eli, M. (2005) Loss of the AP-2alpha transcription factor is associated with the grade of human gliomas. Clin Cancer Res, 11, 267-272.
5. Rao, J. S. (2003) Molecular mechanisms of glioma invasiveness: the role of proteases. Nat Rev Cancer, 3, 489-501.
6. Stamenkovic, I. (2003) Extracellular matrix remodelling: the role of matrix metalloproteinases. J Pathol, 200, 448-464.
7. Binder, D. K. and Berger, M. S. (2002) Proteases and the biology of glioma invasion. J Neurooncol, 56, 149-158.
8. Auge, F., Hornbeck, W. and Laronze, J. Y. (2004) A novel strategy for designing specific gelatinase A inhibitors: potential use to control tumor progression. Crit Rev Oncol Hematol, 49, 277-282.
9. Bachmeier, B. E., Iancu, C. M., Jochum, M. and Nerlich, A. G. (2005) Matrix metalloproteinases in cancer: comparison of known and novel aspects of their inhibition as a therapeutic approach. Expert Rev Anticancer Ther, 5, 149-163.
10. Forget, M. A., Desrosiers, R. R. and Beliveau, R. (1999) Physiological roles of matrix metalloproteinases: implications for tumor growth and metastasis. Can J Physiol Pharmacol, 77, 465-480.
11. Tuerk, C. and Gold, L. (1990) Systematic evolution of ligands by exponential enrichment: RNA ligands to bacteriophage T4 DNA polymerase. Science, 249, 505-510.
12. Ellington, A. D. and Szostak, J. W. (1990) In vitro selection of RNA molecules that bind specific ligands. Nature, 346, 818-822.
13. Robertson, D. L. and Joyce, G. F. (1990) Selection in vitro of an RNA enzyme that specifically cleaves single-stranded DNA. Nature, 344, 467-468.
14. Mayer, G. (2009) The chemical biology of aptamers. Angew Chem Int Ed Engl, 48, 2672-2689.
15. Syed, M. A. and Pervaiz, S. (2010) Advances in aptamers. Oligonucleotides, 20, 215-224.
16. Soontornworajit, B. and Wang, Y. (2011) Nucleic acid aptamers for clinical diagnosis: cell detection and molecular imaging. Anal Bioanal Chem, 399, 1591-1599.
17. Dausse, E., Da Rocha Gomes, S. and Toulmé, J. J. (2009) Aptamers: a new class of oligonucleotides in the drug discovery pipeline? Curr Opin Pharmacol, 9, 602-607.
18. Gomes, S. D. R., Azéma, L., Allard, M. and Toulmé, J. J. (2010) Aptamers as imaging agents. Expert Opinion on Medical Diagnostics, 4, 511-518.
19. Charlton, J., Sennello, J. and Smith, D. (1997) In vivo imaging of inflammation using an aptamer inhibitor of human neutrophil elastase. Chem Biol, 4, 809-816.
20. Lupold, S. E., Hicke, B. J., Lin, Y. and Coffey, D. S. (2002) Identification and characterization of nuclease-stabilized RNA molecules that bind human prostate cancer cells via the prostate-specific membrane antigen. Cancer Res, 62, 4029-4033.
21. Hicke, B. J., Stephens, A. W., Gould, T., Chang, Y. F., Lynott, C. K., Heil, J., Borkowski, S., Hilger, C. S., Cook, G., Warren, S. et al. (2006) Tumor targeting by an aptamer. J Nucl Med, 47, 668-678.
22. Shangguan, D., Li, Y., Tang, Z., Cao, Z. C., Chen, H. W., Mallikaratchy, P., Sefah, K., Yang, C. J. and Tan, W. (2006) Aptamers evolved from live cells as effective molecular probes for cancer study. Proc Natl Acad Sci USA, 103, 11838-11843.
23. Herr, J. K., Smith, J. E., Medley, C. D., Shangguan, D. and Tan, W. (2006) Aptamer-conjugated nanoparticles for selective collection and detection of cancer cells. Anal Chem, 78, 2918-2924.
24. Borbas, K. E., Ferreira, C. S., Perkins, A., Bruce, J. I. and Missailidis, S. (2007) Design and synthesis of mono- and multimeric targeted radiopharmaceuticals based on novel cyclen ligands coupled to anti-MUC1 aptamers for the diagnostic imaging and targeted radiotherapy of cancer. Bioconjug Chem, 18, 1205-1212.
25. Phillips, J. A., Lopez-Colon, D., Zhu, Z., Xu, Y. and Tan, W. (2008) Applications of aptamers in cancer cell biology. Anal Chim Acta, 621, 101-108.
26. Li, W., Yang, X., Wang, K., Tan, W., He, Y., Guo, Q., Tang, H. and Liu, J. (2008) Real-time imaging of protein internalization using aptamer conjugates. Anal Chem, 80, 5002-5008.
27. Sefah, K., Tang, Z. W., Shangguan, D. H., Chen, H., Lopez-Colon, D., Li, Y., Parekh, P., Martin, J., Meng, L., Phillips, J. A. et al. (2009) Molecular recognition of acute myeloid leukemia using aptamers. Leukemia, 23, 235-244.
28. Smith, J. E., Medley, C. D., Tang, Z., Shangguan, D., Lofton, C. and Tan, W. (2007) Aptamer-conjugated nanoparticles for the collection and detection of multiple cancer cells. Anal Chem, 79, 3075-3082.
29. Medley, C. D., Bamrungsap, S., Tan, W. and Smith, J. E. (2011) Aptamer-conjugated nanoparticles for cancer cell detection. Anal Chem, 83, 727-734.
30. Ko, H. Y., Choi, K. J., Lee, C. H. and Kim, S. (2011) A multimodal nanoparticle-based cancer imaging probe simultaneously targeting nucleolin, integrin alphavbeta3 and tenascin-C proteins. Biomaterials, 32, 1130-1138.
31. Zhou, J., Soontornworajit, B. and Wang, Y. (2010) A temperature-responsive antibody-like nanostructure. Biomacromolecules, 11, 2087-2093.
32. Blank, M., Weinschenk, T., Priemer, M. and Schluesener, H. (2001) Systematic evolution of a DNA aptamer binding to rat brain tumor microvessels. selective targeting of endothelial regulatory protein pigpen. J Biol Chem, 276, 16464-16468.
33. Farokhzad, O. C., Jon, S., Khademhosseini, A., Tran, T. N., Lavan, D. A. and Langer, R. (2004) Nanoparticle-aptamer bioconjugates: a new approach for targeting prostate cancer cells. Cancer Res, 64, 7668-7672.
34. Shangguan, D., Meng, L., Cao, Z. C., Xiao, Z., Fang, X., Li, Y., Cardona, D., Witek, R. P., Liu, C. and Tan, W. (2008) Identification of liver cancer-specific aptamers using whole live cells. Anal Chem, 80, 721-728.
35. Li, S., Xu, H., Ding, H., Huang, Y., Cao, X., Yang, G., Li, J., Xie, Z., Meng, Y., Li, X. et al. (2009) Identification of an aptamer targeting hnRNP A1 by tissue slide-based SELEX. J Pathol, 218, 327-336.
36. Zhang, Z., Guo, L., Guo, A., Xu, H., Tang, J., Guo, X. and Xie, J. (2010) In vitro lectin-mediated selection and characterization of rHuEPO-alpha-binding ssDNA aptamers. Bioorg Med Chem, 18, 8016-8025.
37. Shi, H., He, X., Wang, K., Wu, X., Ye, X., Guo, Q., Tan, W., Qing, Z., Yang, X. and Zhou, B. (2011) Activatable aptamer probe for contrast-enhanced in vivo cancer imaging based on cell membrane protein-triggered conformation alteration. Proc Natl Acad Sci USA, 108, 3900-3905.
38. Sousa, R. and Padilla, R. (1995) A mutant T7 RNA polymerase as a DNA polymerase. Embo J, 14, 4609-4621.
39. Bartel, D. P. and Szostak, J. W. (1994) In Nagai, K. and Mattaj, I. W. (eds.), RNA-Protein interactions. IRL Press, Oxford, pp. 248-268.
40. McEntee, K., Weinstock, G. M. and Lehman, I. R. (1980) recA protein-catalysed strand assimilation: stimulation by Escherichia coli single-stranded DNA-binding protein. Proc. Natl. Acad. Sci. USA, 77, 857-861.
41. Winnard, P., Jr., Chang, F., Rusckowski, M., Mardirossian, G. and Hnatowich, D. J. (1997) Preparation and use of NHS-MAG3 for technetium-99m labeling of DNA. Nucl Med Biol, 24, 425-432.
42. Di Primo, C., Dausse, E. and Toulmé, J. J. (2011) Surface plasmon resonance investigation of RNA aptamer-RNA ligand interactions. Methods Mol Biol, 764, 279-300.
43. Comincini, S., Paolillo, M., Barbieri, G., Palumbo, S., Sbalchiero, E., Azzalin, A., Russo, M. A. and Schinelli, S. (2009) Gene expression analysis of an EGFR indirectly related pathway identified PTEN and MMP9 as reliable diagnostic markers for human glial tumor specimens. J Biomed Biotechnol, 2009, 924565.
44. Das, G., Shiras, A., Shanmuganandam, K. and Shastry, P. (2010) Rictor regulates MMP-9 activity and invasion through Raf-1-MEK-ERK signaling pathway in glioma cells. Mol Carcinog, 50, 412-423.
45. Lee, E. J., Kim, S. Y., Hyun, J. W., Min, S. W., Kim, D. H. and Kim, H. S. (2010) Glycitein inhibits glioma cell invasion through down-regulation of MMP-3 and MMP-9 gene expression. Chem Biol Interact, 185, 18-24.
46. Veeravalli, K. K., Chetty, C., Ponnala, S., Gondi, C. S., Lakka, S. S., Fassett, D., Klopfenstein, J. D., Dinh, D. H., Gujrati, M. and Rao, J. S. (2010) MMP-9, uPAR and cathepsin B silencing downregulate integrins in human glioma xenograft cells in vitro and in vivo in nude mice. PLoS One, 5, e11583.
47. Bremer, C., Tung, C. H. and Weissleder, R. (2001) In vivo molecular target assessment of matrix metalloproteinase inhibition. Nat Med, 7, 743-748.
48. Wallis de Vries, B. M., Hillebrands, J. L., van Dam, G. M., Tio, R. A., de Jong, J. S., Slart, R. H. and Zeebregts, C. J. (2009) Images in cardiovascular medicine. Multispectral near-infrared fluorescence molecular imaging of matrix metalloproteinases in a human carotid plaque using a matrix-degrading metalloproteinase-sensitive activatable fluorescent probe. Circulation, 119, e534-536.
49. Furumoto, S., Takashima, K., Kubota, K., Ido, T., Iwata, R. and Fukuda, H. (2003) Tumor detection using 18F-labeled matrix metalloproteinase-2 inhibitor. Nucl Med Biol, 30, 119-125.
50. Razavian, M., Zhang, J., Nie, L., Tavakoli, S., Razavian, N., Dobrucki, L. W., Sinusas, A. J., Edwards, D. S., Azure, M. and Sadeghi, M. M. (2010) Molecular imaging of matrix metalloproteinase activation to predict murine aneurysm expansion in vivo. J Nucl Med, 51, 1107-1115.
51. Scherer, R. L., VanSaun, M. N., McIntyre, J. O. and Matrisian, L. M. (2008) Optical imaging of matrix metalloproteinase-7 activity in vivo using a proteolytic nanobeacon. Mol Imaging, 7, 118-131.
52. Sprague, J. E., Li, W. P., Liang, K., Achilefu, S. and Anderson, C. J. (2006) In vitro and in vivo investigation of matrix metalloproteinase expression in metastatic tumor models. Nucl Med Biol, 33, 227-237.
53. Ujula, T., Huttunen, M., Luoto, P., Perakyla, H., Simpura, I., Wilson, I., Bergman, M. and Roivainen, A. (2010) Matrix metalloproteinase 9 targeting peptides: syntheses, 68Ga-labeling, and preliminary evaluation in a rat melanoma xenograft model. Bioconjug Chem, 21, 1612-1621.
54. Kuge, Y., Takai, N., Ogawa, Y., Temma, T., Zhao, Y., Nishigori, K., Ishino, S., Kamihashi, J., Kiyono, Y., Shiomi, M. et al. (2010) Imaging with radiolabelled anti-membrane type 1 matrix metalloproteinase (MT1-MMP) antibody: potentials for characterizing atherosclerotic plaques. Eur J Nucl Med Mol Imaging, 37, 2093-2104.

1. A nucleic acid aptamer that binds specifically to human matrix metalloproteinase 9 (hMMP-9) characterized in that said nucleic acid comprises the following nucleotide sequence:
5′-NS1-CCU-NS3-N1-NS5-CCCGUUAGCC-NS6-N2-NS4-
CCC-NS2-3′
wherein
NS1 and NS2 consist of polynucleotides having 1, 2 or 3 nucleotides in length, and NS1 and NS2 have complementary sequences;
NS3 and NS4 consist of polynucleotides having 2 nucleotides in length, and NS1 and NS2 have complementary sequences
N1 and N2 consist of a nucleotide, and N1 is or is not complementary to N2
NS5 and NS6 consist of polynucleotides having 4 nucleotides in length, and NS5 and NS6 have complementary sequences.

2. The nucleic acid aptamer according to claim 1 wherein NS1 represents C, GC, UGC or ACG and NS2 represents G, GC, GCA or CGU respectively.

3. The nucleic acid aptamer according to claim 1 wherein NS3 and NS4 represent GC or CG.

4. The nucleic acid aptamer according to claim 1 wherein N1 and N2 represent C or A, or N1 represents C and N2 represents G.

5. The nucleic acid aptamer according to claim 1 wherein NS5 represents CUCA or GAGU and NS6 represents UGAG or ACUC respectively.

6. The nucleic acid aptamer according to claim 1 which comprises or consists of a nucleic acid sequence selected from the group consisting of SEQ ID NO: 1-7.

7. The nucleic acid aptamer according to claim 1 which comprises or consists of a nucleic acid sequence as set forth in SEQ ID NO:8.

8. The nucleic acid aptamer according to claim 1 wherein the pyrimidines are replaced by 2′-fluoropyrimidines.

9. The nucleic acid aptamer according to claim 1 wherein the purine ribonucleosides are substituted by 2′O-methyl residues.

10. An imaging agent having high affinity and specificity for hMMP-9, said imaging agent comprising at least one hMMP-9 nucleic acid aptamer moiety comprising the following nucleotide sequence:
5′-NS1-CCU-NS3-N1-NS5-CCCGUUAGCC-NS6-N2-NS4-
CCC-NS2-3′
wherein
NS1 and NS2 consist of polynucleotides having 1, 2 or 3 nucleotides in length, and NS1 and NS2 have complementary sequences;
NS3 and NS4 consist of polynucleotides having 2 nucleotides in length, and NS1 and NS2 have complementary sequences
N1 and N2 consist of a nucleotide, and N1 is or is not complementary to N2
NS5 and NS6 consist of polynucleotides having 4 nucleotides in length, and NS5 and NS6 have complementary sequences;
and wherein said hMMP-9 nucleic acid aptamer moiety is associated with at least one detectable moiety.

11. The imaging agent according to claim 10 wherein the detectable moiety is detectable by an imaging technique selected from the group consisting of ultrasonography, Magnetic Resonance Imaging (MRI), Positron Emission Tomography (PET), Single Photon Emission Computed Tomography (SPECT), fluorescence spectroscopy, Computed Tomography, X-ray radiography, or any combination of these techniques.

12. The imaging agent according to claim 11 wherein the detectable moiety is selected from the group consisting of radionuclides, paramagnetic metal ions ultrasmall superparamagnetic iron oxides and fluorescent moieties.

13. The imaging agent according to claim 10 which consists of a nucleic acid sequence as set forth in SEQ ID NO: 3 wherein the pyrimidines are 2′-fluoropyrimidine and the purine ribonucleosides are substituted by 2′0-methyl residues and which is conjugated at its 5′ end to S-acetylmercaptoacetyltriglycine (MAG3) through a hexylamino linker and labeled with 99mTc.

14. A method for imaging hMMP9 in a subject in need thereof comprising
administering to said subject an imaging agent having high affinity and specificity for hMMP-9, said imaging agent comprising at least one hMMP-9 nucleic acid aptamer moiety comprising the following nucleotide sequence:
5′-NS1-CCU-NS3-N1-NS5-CCCGUUAGCC-NS6-N2-NS4-
CCC-NS2-3′
wherein
NS1 and NS2 consist of polynucleotides having 1, 2 or 3 nucleotides in length, and NS1 and NS2 have complementary sequences;
NS3 and NS4 consist of polynucleotides having 2 nucleotides in length, and NS1 and NS2 have complementary sequences
N1 and N2 consist of a nucleotide, and N1 is or is not complementary to N2
NS5 and NS6 consist of polynucleotides having 4 nucleotides in length, and NS5 and NS6 have complementary sequences;
and wherein said hMMP-9 nucleic acid aptamer moiety is associated with at least one detectable moiety that allows visualization of the imaging agent using imaging techniques; and
visualizing the imaging agent in the subject using imaging techniques.

15. The method according to claim 14 wherein said subject suffers from a disease selected from the group consisting of inflammation, a neurovascular or neurodegenerative disease atherosclerosis and cancers.

16. The method of claim 15, wherein said neurovascular or neurodegenerative disease is brain injury, stroke, or hemorrhagic transformation.


SEQUENCE LISTING (22 sequences)

SEQ ID NO: 1 - RNA, 32 nt, Artificial
  Synthetic nucleotide sequence of aptamer F3C1
cccugcccucacccguuagccugagcgccccg

SEQ ID NO: 2 - RNA, 34 nt, Artificial
  Synthetic nucleotide sequence of aptamer F3B′
gcccugcccucacccguuagccugagcgccccgc

SEQ ID NO: 3 - RNA, 36 nt, Artificial
  Synthetic nucleotide sequence of aptamer F3B
ugcccugcccucacccguuagccugagcgccccgca

SEQ ID NO: 4 - RNA, 36 nt, Artificial
  Synthetic nucleotide sequence of aptamer F3BAA
ugcccugcacucacccguuagccugagagccccgca

SEQ ID NO: 5 - RNA, 36 nt, Artificial
  Synthetic nucleotide sequence of aptamer F3BCG
ugcccugcccucacccguuagccugagggccccgca

SEQ ID NO: 6 - RNA, 36 nt, Artificial
  Synthetic nucleotide sequence of F3Binv1
ugcccugccgagucccguuagccacuccgccccgca

SEQ ID NO: 7 - RNA, 36 nt, Artificial
  Synthetic nucleotide sequence of aptamer F3Binv2
acgccucgccucacccguuagccugagccgccccgu

SEQ ID NO: 8 - RNA, 68 nt, Artificial
  Synthetic nucleotide sequence of aptamer F3
gguuaccagccuucacugcccugcccucacccguuagccugagcgccccgcaccacggucggucacac

SEQ ID NO: 9 - DNA, 68 bp, Artificial
  Synthetic oligonucleotide primer
gtgtgaccgaccgtggtgcnnnnnnnnnnnnnnnnnnnnnnnnnnnnnngcagtgaaggctggtaacc

SEQ ID NO: 10 - DNA, 19 bp, Artificial
  Synthetic oligonucleotide primer
gtgtgaccgaccgtggtgc

SEQ ID NO: 11 - DNA, 36 bp, Artificial
  Synthetic oligonucleotide primer
taatacgactcactataggttaccagccttcactgc

SEQ ID NO: 12 - RNA, 36 nt, Artificial
  Synthetic oligonucleotide control sequence
ugccaaacgcguccccuuugcccggccuccgccgca

SEQ ID NO: 13 - RNA, 2 nt, Artificial
  Synthetic nucleotide sequence of NS1
gc

SEQ ID NO: 14 - RNA, 3 nt, Artificial
  Synthetic nucleotide sequence of NS1
ugc

SEQ ID NO: 15 - RNA, 3 nt, Artificial
  Synthetic nucleotide sequence of NS1
acg

SEQ ID NO: 16 - RNA, 3 nt, Artificial
  Synthetic nucleotide sequence of NS2
gca

SEQ ID NO: 17 - RNA, 3 nt, Artificial
  Synthetic nucleotide sequence of NS2
cgu

SEQ ID NO: 18 - RNA, 2 nt, Artificial
  Synthetic nucleotide sequence of NS4
cg

SEQ ID NO: 19 - RNA, 4 nt, Artificial
  Synthetic nucleotide sequence of NS5
cuca

SEQ ID NO: 20 - RNA, 4 nt, Artificial
  Synthetic nucleotide sequence of NS5
gagu

SEQ ID NO: 21 - RNA, 4 nt, Artificial
  Synthetic nucleotide sequence of NS6
ugag

SEQ ID NO: 22 - RNA, 4 nt, Artificial
  Synthetic nucleotide sequence of NS6
acuc